(19) United States
(12) Patent Application Publication
(10) Pub. No.: US 2007/0129382 A1
(43) Pub. Date: Jun. 7, 2007

(54) Crf receptor antagonists, their preparations, their pharmaceutical composition, and their uses

(76) Inventors: Dimitri Grigoriadis, San Diego, CA (US); Deborah Slee, Cardiff, CA (US); John Williams, San Diego, CA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/597,788

(22) PCT Filed: Feb. 9, 2005
(86) PCT No.: PCT/GB05/00432

§ 371 Date: Aug. 8, 2006

Related U.S. Application Data
(60) Provisional application No. 60/544,758, filed on Feb. 14, 2004.

(51) Int. Cl. A61K 31/519; C07D 487/04

(52) U.S. Cl. 514/259.3; 544/281

(57) ABSTRACT

CRF receptor antagonists are disclosed which have utility in the treatment of a variety of disorders, including the treatment of disorders manifesting hypersecretion of CRF in warm-blooded animals. CRF receptor antagonists which are labeled with a positron emitting isotope for use in PET are also disclosed.

### TECHNICAL FIELD

This invention relates generally to CRF receptor antagonists their preparation, their pharmaceutical composition and their uses. CRF receptor antagonists are disclosed which have utility in the treatment of a variety of disorders, including the treatment of disorders manifesting hypersecretion of CRF in a warm-blooded animals. CRF receptor antagonists which are labeled with a positron emitting isotope for use in PET are also disclosed.

### BACKGROUND OF THE INVENTION

Positron Emission Tomography (PET) is a non-invasive imaging technology where a compound labeled with a positron-emitting isotope is administered to provide an in-situ image of the binding of the positron-emitting compound. The image can be used to determine localization and quantification of specific areas where the labeled compound binds providing diagnostic and drug discovery applications. Positron emitting isotopes generally used for PET include ¹¹C, ¹³N, ¹⁵O, ¹⁸F, ⁷⁶Br and ¹²⁴I. The ¹³N and ¹⁵O isotopes have very short half lives limiting their usefulness. ⁷⁶Br and ¹²⁴I have half lives of 16.3 hours and 4.2 days respectively which would allow easy production of labeled CRF antagonists but the addition of the large halogen group changes the activity of the compound and adds unwanted lipophilicity. ¹¹C has a relatively short 20.5 minute half life but ease of incorporating ¹¹C into a PET compound makes this a useful isotope. ¹⁸F has a half life of 110 minutes which allows sufficient time for incorporation of the isotope into a tracer, purification and administration into a subject.

The incorporation of a positron emitting isotope into a Corticotropin-releasing factor (CRF) antagonist may allow for the use of PET imaging in determining the binding of labeled antagonists to CRF receptors. CRF is a 41 amino acid peptide which has been found to produce profound alterations in endocrine, nervous and immune system function. CRF is believed to be the major physiological regulator of the basal and stress-release of adrenocorticotropic hormone (“ACTH”), β-endorphin, and other pro-opiomelanocortin (“POMC”)-derived peptides from the anterior pituitary (Vale et al., Science 213:1394-1397, 1981). The CRF receptor is coupled to a GTP-binding protein (Perrin et al., Endocrinology 118:1171-1179, 1986) which mediates CRF-stimulated increase in intracellular production of cAMP (Bilezikjian, L. M., and W. W. Vale, Endocrinology 113:657-662, 1983). In addition to its role in stimulating the production of ACTH and POMC, CRF is also believed to coordinate many of the endocrine, autonomic, and behavioral responses to stress, and may be involved in the pathophysiology of affective disorders. Moreover, CRF is believed to be a key intermediary in communication between the immune, central nervous, endocrine and cardiovascular systems (Crofford et al., J. Clin. Invest. 90:2555-2564, 1992; Sapolsky et al., Science 238:522-524, 1987; Tilders et al., Regul. Peptides 5:77-84, 1982). Overall, CRF appears to be one of the pivotal central nervous system neurotransmitters and plays a crucial role in integrating the body's overall response to stress.

Administration of CRF directly to the brain elicits behavioral, physiological, and endocrine responses identical to those observed for an animal exposed to a stressful environment. For example, intracerebroventricular injection of CRF results in behavioral activation (Sutton et al., Nature 297:331, 1982), persistent activation of the electroencephalogram (Ehlers et al., Brain Res. 278:332, 1983), stimulation of the sympathoadrenomedullary pathway (Brown et al., Endocrinology 110:928, 1982), an increase of heart rate and blood pressure (Fisher et al., Endocrinology 110:2222, 1982), an increase in oxygen consumption (Brown et al., Life Sciences 30:207, 1982), alteration of gastrointestinal activity (Williams et al., Am. J. Physiol. 253:G582, 1987), suppression of food consumption (Levine et al., Neuropharmacology 22:337, 1983), modification of sexual behavior (Sirinathsinghji et al., Nature 305:232, 1983), and immune function compromise (Irwin et al., Am. J. Phlysiol. 255:R744, 1988). Furthermore, clinical data suggests that CRF may be hypersecreted in the brain in depression, anxiety-related disorders, and anorexia nervosa. (DeSouza, Ann. Reports in Med. Chem. 25:215-223, 1990). Accordingly, clinical data suggests that CRF receptor antagonists may represent novel antidepressant and/or anxiolytic drugs that may be useful in the treatment of the neuropsychiatric disorders manifesting hypersecretion of CRF.

The administration of CRF antagonist which has been labeled with a positron emitting isotope may permit the use of PET imaging to provide an in situ evaluation of the labeled antagonist's binding characteristics in the brain. The antagonist should possess several characteristics such as specific binding to CRF receptors, high brain penetration and the ability to be labeled with a positron emitting isotope as the ultimate step in the synthesis.

U.S. Pat. Nos. 6,514,982, 6,531,475, 6,664,261 and PCT application WO98/03510 describe CRF antagonists for use in treating various CRF mediated diseases. The synthesis of certain pyrrolopyrimidines as PET ligands for the CRF receptor has also been described. (L. Martarello et al., Nuclear Medicine and Biology, 28 (2001) 187-195).

While significant strides have been made toward achieving CRF regulation through administration of CRF receptor antagonists, there remains a need in the art for effective small molecule CRF receptor antagonists. There is also a need for pharmaceutical compositions containing such CRF receptor antagonists, as well as methods relating to the use thereof to treat, for example, stress-related disorders. The used of CRF antagonists bearing a Positron emitting isotope as diagnostic and drug discovery tools is also addressed. The present invention fulfills these needs, and provides other related advantages.

### SUMMARY OF THE INVENTION

In brief, this invention is generally directed to CRF receptor antagonists 2,5-dimethyl-3-(2,4-dimethoxyphenyl)-7-(diethylamino)pyrazolo[2,3-a]pyrimidine, 2,5-dimethyl-3-(2,4-dimethoxyphenyl)-7-(N-ethyl-N-methoxyethylamino)pyrazolo[2,3-a]pyrimidine, 2,5-dimethyl-3-(2,4-dimethoxyphenyl)-7-{2-(S)-methoxymethylpyrrolidinyl}-pyrazolo[2,3-a]pyrimidine, 2,5-dimethyl-3-(2,4-dimethoxyphenyl)-7-(N-ethyl-N-{2-fluoroethyl}amino)pyrazolo[2,3-a]pyrimidine and 6-(cyclopropylmethyl)-2-(2,4-dichlorophenyl)-7-(S)-ethyl-7,8-dihydro-4-methyl-6H-1,3,6,8a-tetraazaacenaphthylene, including stereoisomers, prodrugs and pharmaceutically acceptable salts thereof.

The CRF receptor antagonists of this invention may be radiolabeled and have utility as diagnostic agents, research agents and agents useful in drug development and evaluation. These and other aspects of the invention will be apparent upon reference to the following detailed description. To this end, various references are set forth herein which describe in more detail certain procedures, compounds and/or compositions, and are hereby incorporated by reference in their entirety.

### DETAILED DESCRIPTION OF THE INVENTION

The present invention is directed generally to compounds useful as corticotropin-releasing factor (CRF) receptor antagonists and as PET ligands.

In a first embodiment, the CRF receptor antagonists of this invention are taken from 2,5-dimethyl-3-(2,4-dimethoxyphenyl)-7-(diethylamino)pyrazolo[2,3-a]pyrimidine, 2,5-dimethyl-3-(2,4-dimethoxyphenyl)-7-(N-ethyl-N-methoxyethylamino)pyrazolo[2,3-a]pyrimidine, 2,5-dimethyl-3-(2,4-dimethoxyphenyl)-7-{2-(S)-methoxymethylpyrrolidinyl}-pyrazolo[2,3-a]pyrimidine, 2,5-dimethyl-3-(2,4-dimethoxyphenyl)-7-(N-ethyl-N-{2-fluoroethyl}amino)pyrazolo[2,3-a]pyrimidine and 6-(cyclopropylmethyl)-2-(2,4-dichlorophenyl)-7-(S)-ethyl-7,8-dihydro-4-methyl-6H-1,3,6,8a-tetraazaacenaphthylene.

In another embodiment, the CRF antagonist is labeled with a positron emitting isotope to allow for imaging of the antagonist by PET. The isotope is generally taken from ¹¹C and ¹⁸F as these isotopes may generally be added as the last step of a synthesis, may be purified quickly and may be administered to a subject, generally in intravenous form.

The compounds of the present invention may generally be utilized as the free base. Alternatively, the compounds of this invention may be used in the form of acid addition salts. Acid addition salts of the free base amino compounds of the present invention may be prepared by methods well known in the art, and may be formed from organic and inorganic acids. Suitable organic acids include maleic, fumaric, benzoic, ascorbic, succinic, methanesulfonic, acetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Thus, the term “pharmaceutically acceptable salt” of structure (I) is intended to encompass any and all acceptable salt forms.

In general, the compounds of the present invention may be made according to the organic synthesis techniques known to those skilled in this field, as well as by the representative methods set forth in the Examples.

The effectiveness of a compound as a CRF receptor antagonist may be determined by various assay methods. Suitable CRF antagonists of this invention are capable of inhibiting the specific binding of CRF to its receptor and antagonizing activities associated with CRF. A compound of the present invention may be assessed for activity as a CRF antagonist by one or more generally accepted assays for this purpose, including (but not limited to) the assays disclosed by DeSouza et al. (J. Neuroscience 7:88, 1987) and Battaglia et al. (Synapse 1:572, 1987). As mentioned above, suitable CRF antagonists include compounds which demonstrate CRF receptor affinity. CRF receptor affinity may be determined by binding studies that measure the ability of a compound to inhibit the binding of a radiolabeled CRF (e.g., [¹²⁵I]tyrosine-CFR) to its receptor (e.g., receptors prepared from rat cerebral cortex membranes). The radioligand binding assay described by DeSouza et al. (supra, 1987) provides an assay for determining a compound's affinity for the CRF receptor. Such activity is typically calculated from the IC₅₀ as the concentration of a compound necessary to displace 50% of the radiolabeled ligand from the receptor, and is reported as a “Kᵢ” value calculated by the following equation:

$${K}_{i} = \frac{{IC}_{50}}{1 + L / {K}_{D}}$$

where L=radioligand and K_{D}=affinity of radioligand for receptor (Cheng and Prusoff, Biochem. Pharmacol. 22:3099, 1973).

In addition to inhibiting CRF receptor binding, a compound's CRF receptor antagonist activity may be established by the ability of the compound to antagonize an activity associated with CRF. For example, CRF is known to stimulate various biochemical processes, including adenylate cyclase activity. Therefore, compounds may be evaluated as CRF antagonists by their ability to antagonize CRF-stimulated adenylate cyclase activity by, for example, measuring cAMP levels. The CRF-stimulated adenylate cyclase activity assay described by Battaglia et al. (supra, 1987) provides an assay for determining a compound's ability to antagonize CRF activity. Accordingly, CRF receptor antagonist activity may be determined by assay techniques which generally include an initial binding assay (such as disclosed by DeSouza (supra, 1987)) followed by a cAMP screening protocol (such as disclosed by Battaglia (supra, 1987)).

The CRF receptor antagonists of the present invention demonstrate activity at the CRF receptor site, and may be used as therapeutic agents for the treatment of a wide range of disorders or illnesses including endocrine, psychiatric, and neurological disorders or illnesses. More specifically, the CRF receptor antagonists of the present invention may be useful in treating physiological conditions or disorders arising from the hypersecretion of CRF. Because CRF is believed to be a pivotal neurotransmitter that activates and coordinates the endocrine, behavioral and automatic responses to stress, the CRF receptor antagonists of the present invention can be used to treat neuropsychiatric disorders. Neuropsychiatric disorders which may be treatable by the CRF receptor antagonists of this invention include affective disorders such as depression; anxiety-related disorders such as generalized anxiety disorder, panic disorder, obsessive-compulsive disorder, abnormal aggression, cardiovascular abnormalities such as unstable angina and reactive hypertension; and feeding disorders such as anorexia nervosa, bulimia, and irritable bowel syndrome. CRF antagonists may also be useful in treating stress-induced immune suppression associated with various diseases states, as well as stroke. Other uses of the CRF antagonists of this invention include treatment of inflammatory conditions (such as rheumatoid arthritis, uveitis, asthma, inflammatory bowel disease and G.I. motility), pain, Cushing's disease, infantile spasms, epilepsy and other seizures in both infants and adults, and various substance abuse and withdrawal (including alcoholism).

Additional uses of the PET labeled CRF receptor antagonists of the present invention include use in clinical research and diagnosis of various disease states involving the CRF receptor. The labeled CRF receptor antagonists may also useful in the study of pharmacology, drug pharmacokinetics, pharmacodynamics, biodistribution and metabolism (Victor Pike, Drug Information Journal, 31 (1997), 997-1013).

In another embodiment of the invention, pharmaceutical compositions containing one or more CRF receptor antagonists are disclosed. For the purposes of administration, the compounds of the present invention may be formulated as pharmaceutical compositions. Pharmaceutical compositions of the present invention comprise a CRF receptor antagonist of the present invention (i.e., a compound of structure (I)) and a pharmaceutically acceptable carrier and/or diluent. The CRF receptor antagonist is present in the composition in an amount which is effective to treat a particular disorder—that is, in an amount sufficient to achieve CRF receptor antagonist activity, and preferably with acceptable toxicity to the patient. Preferably, the pharmaceutical compositions of the present invention may include a CRF receptor antagonist in an amount from 0.1 mg to 250 mg per dosage depending upon the route of administration, and more preferably from 1 mg to 60 mg. Appropriate concentrations and dosages can be readily determined by one skilled in the art.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets which contain, in addition to a CRF receptor antagonist, diluents, dispersing and surface active agents, binders, and lubricants. One skilled in this art may further formulate the CRF receptor antagonist in an appropriate manner, and in accordance with accepted practices, such as those disclosed in Remington's Pharmaceutical Sciences, Gennaro, Ed., Mack Publishing Co., Easton, Pa. 1990.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound.

In another embodiment, the present invention provides a method for treating a variety of disorders or illnesses, including endocrine, psychiatric and neurological disorders or illnesses. Such methods include administering of a compound of the present invention to a warm-blooded animal in an amount sufficient to treat the disorder or illness. Such methods include systemic administration of a CRF receptor antagonist of this invention, preferably in the form of a pharmaceutical composition. As used herein, systemic administration includes oral and parenteral methods of administration. For oral administration, suitable pharmaceutical compositions of CRF receptor antagonists include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parental administration, the compounds of the present invention can be prepared in aqueous injection solutions which may contain, in addition to the CRF receptor antagonist, buffers, antioxidants, bacteriostats, and other additives commonly employed in such solutions.

As mentioned above, administration of a compound of the present invention can be used to treat a wide variety of disorders or illnesses. In particular, the compounds of the present invention may be administered to a warm-blooded animal for the treatment of depression, anxiety disorder, panic disorder, obsessive-compulsive disorder, abnormal aggression, unstable angina, reactive hypertension, anorexia nervosa, bulimia, irritable bowel syndrome, stress-induced immune suppression, stroke, inflammation, pain, Cushing's disease, infantile spasms, epilepsy, and substance abuse or withdrawal.

The following examples are provided for purposes of illustration, not limitation.

### EXAMPLES

The CRF receptor antagonists of this invention may be prepared by the methods disclosed in Examples 1 to 4. Example 5 presents a method for determining the receptor binding affinity.

Analytical HPLC-MS (LC-MS)

HP 1100 series: equipped with an auto-sampler, an UV detector (220 nM and 254 nM), a MS detector (electrospray);

HPLC column: YMC ODS AQ, S-5, 5μ, 2.0×50 mm cartridge;

HPLC gradients: 1.5 mL/minute, from 10% acetonitrile in water to 90% acetonitrile in water in 2.5 minutes, maintaining 90% for 1 minute.

Prep. HPLC-MS

Gilson HPLC-MS equipped with Gilson 215 auto-sampler/fraction collector, an UV detector and a ThermoFinnigan AQA Single QUAD Mass detector (electrospray);

HPLC column: BHK ODS-O/B, 5μ, 30×75 mm

HPLC gradients: 35 mL/minute, 10% acetonitrile in water to 100% acetonitrile in 7 minutes, maintaining 100% acetonitrile for 3 minutes.

### Example 1

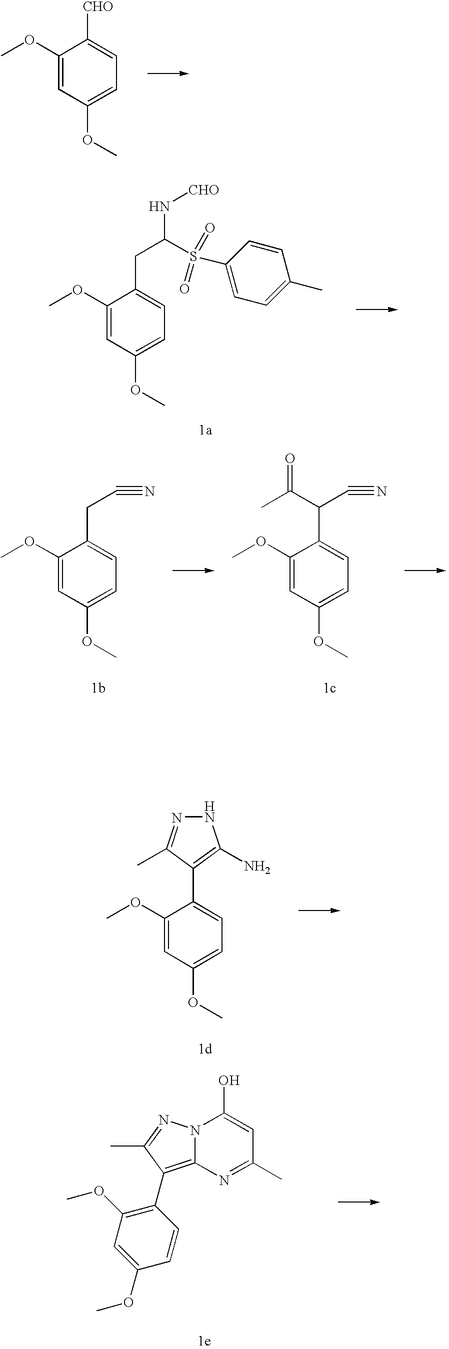 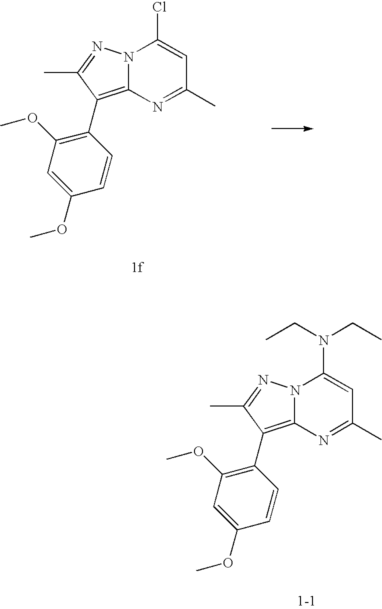 Step 1A:

A suspension of potassium t-butyloxide (7.3 g, 65 mmol, 1.4 eq) in 1,2-dimethoxyethane (DME, 40 mL) was chilled to −50° C. under nitrogen. Tosylmethyl isocyanide (9.1 g, 46.5 mmol, 1 eq) in 40 mL DME was added dropwise while the temperature was kept below −5020 C. 2,4-Dimethoxybenzaldehyde (7.7 g, 46.5 mmol, 1 eq) was added dropwise and the reaction mixture was stirred for 30 minutes to give compound 1a. MeOH (100 mL) was added and the mixture was refluxed for 30 minutes. Most of the DME and MeOH were removed, the residue was resuspended in water (100 mL) and ethyl acetate (100 mL). Following neutralization with acetic acid, the organic layer was washed with brine and dried under sodium sulfate, concentrated and purified by silica gel column using 25% ethyl acetate in hexane to yield 5.5 g of 1b as a white solid.

Step 1B:

To 2,4-dimethoxyphenylacetonitrile 1b (36.0 g, 204 mmol, 1 equiv.) in dry THF (300 mL) was added 12 g (510 mmol, 2.5 equiv) of 60% NaH portionwise. A few drops of EtOAc were added and the reaction was heated gently to initiate the reaction. The remaining EtOAc (150 mL) was then added dropwise. After 2 hours the reaction was poured into ice water and was washed with ether. The aqueous layer was acidified to pH=6 and was extracted with EtOAc, dried over MgSO₄, filtered and concentrated in vacuo to give 44.2 g of 1c.

Step 1C:

A mixture of 1c (8.17 g, 37.3 mmol) and hydrazine monohydrobromide (15.3 g, 135.4 mmol) was refluxed in EtOH/H₂O (6:1) for 5 h. After evaporation of EtOH, the residue was extracted with EtOAc and water. The organic phase was dried and evaporated to dryness to give 1d.

Step 1D:

A mixture of the compound 1d (6.5 g, 27.9 mmol) was refluxed with ethyl acetoacetate (5.0 mL) in acetic acid (100 mL) for 3 h. After evaporation of AcOH, ethyl ether was added and 1e (10.4 g) was obtained after filtration through a medium fritted funnel. In place of the acetic acid solvent, the reaction may be refluxed in dioxane (50 mL) overnight followed by addition of ether to precipitate the product.

Step 1E:

To a suspension of compound 1e (1.9 g, 6.3 mmol) in acetonitrile was added POCl₃ (2.2 mL, 24.1 mmol). The suspension was heat to reflux for 5 h. The resulting orange solution was cooled to room temperature and poured onto ice-water. After extraction with EtOAc, compound 1f was obtained by column chromatography purification (1.70 g, 85%).

Step 1F:

Compound 1f (30 mg) and excess diethylamine in acetonitrile (0.8 mL) were heated at 160° C. with a microwave for 16 min. Purification with a Sciex2 prep LC-MS system gave 2,5-dimethyl-3-(2,4-dimethoxyphenyl)-7-(diethylamino)pyrazolo[2,3-a]pyrimidine 1-1. LCMS 355 (MH⁺), FW=354.45

Following the same general procedure the following compounds were also synthesized:
2,5-dimethyl-3-(2,4-dimethoxyphenyl)-7-(N-ethyl-N-methoxyethylamino)pyrazolo[2,3-5 a]pyrimidine. LCMS 385 (MH⁺), FW=384.48;
2,5-dimethyl-3-(2,4-dimethoxyphenyl)-7-{2-(S)-methoxymethylpyrrolidinyl}-pyrazolo[2,3-a]pyrimidine. LCMS 396 (MH⁺), FW=396.49.

### Example 2

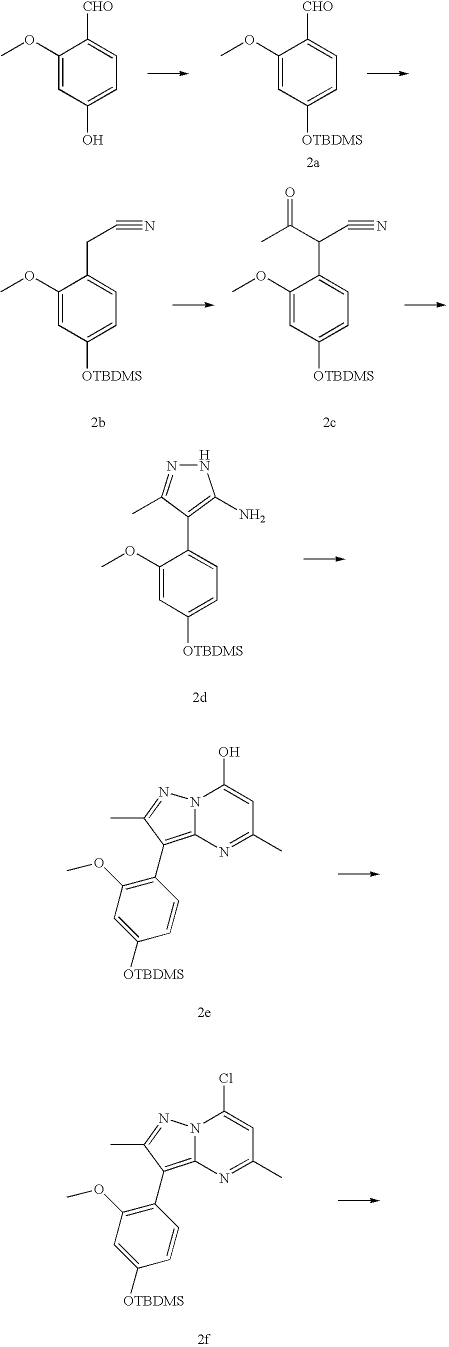 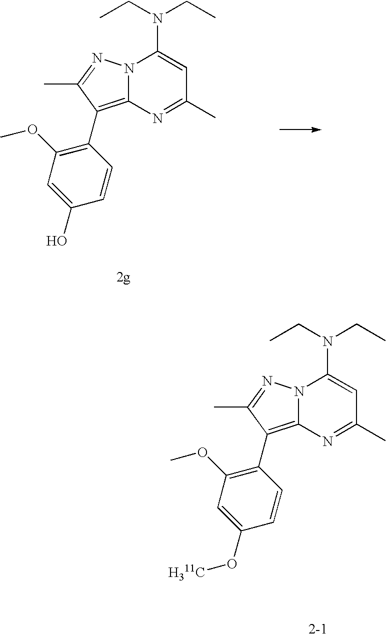 Step 2A:

2-Hydroxy-4-methoxybenzaldehyde is protected by reaction with tert-butyldimethylsilyl chloride (TBDMSCl) and imidazole in DMF to give the tert-butyldimethylsilyl ether 2a. Using 2a and following the procedure as outlined in steps 1A to 1E, compound 2f is realized. 2f and diethylamine following the procedure of Step 1F followed by deprotection of the tert-butyldimethylsilyl group with tetrabutylammonium fluoride or acid under standard conditions gives 2g. Alkylation of the hydroxy group of 2g with ¹¹C methyl iodide and sodium hydride in solvent such as DMF or acetonitrile gives 2-1.

### Example 3

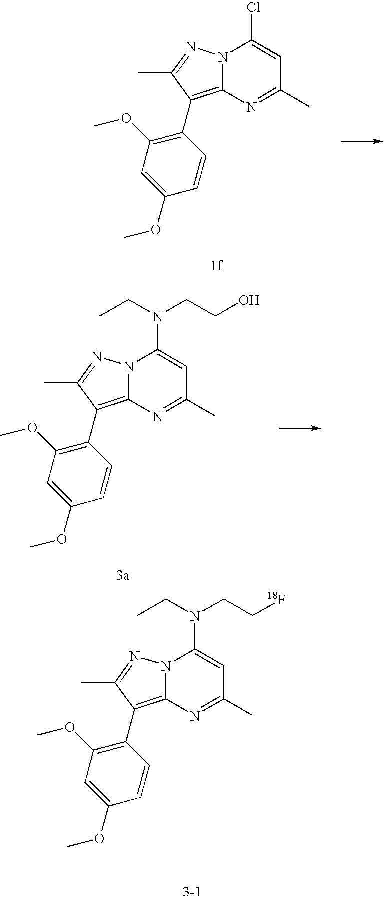 Step 3A:

Compound 1f and ethylaminoethanol following the procedure of Step 1F gives 3a.

Step 3B:

Compound 3a is converted to the sulfonate ester such as mesylate, tosylate or triflate followed by reaction with ¹⁸F ion to give 3-1 (L. Martarello et al., Nuclear Medicine and Biology 28 (2001) 187-195). Compound 3a and methanesulfonyl chloride in pyridine yields a mesylate which undergoes a nucleophilic substitution with K¹⁸F to give 3-1 which is purified by HPLC.

### Example 4

### 6-(Cycloprophylmethyl)-2-(2,4-Dichlorophenyl)-7-(S)-Ethyl-7,8-Dihydro-4-Methyl-6H-1,3,6,8A-Tetraazaacenaphthylene

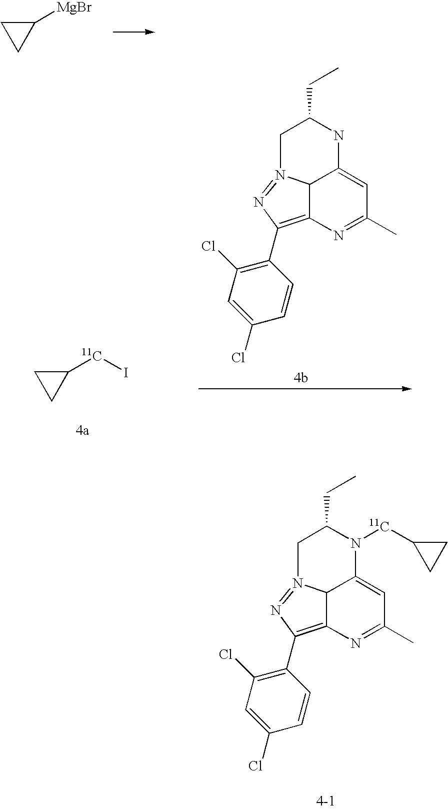 Step 4A:

According to the general procedure of Ishiwata et al., Appl. Radiat. Isot., 46, 10: 1009-1013,cyclopropyl-[¹¹C]-methyliodide is prepared. [¹¹C]CO₂ is transferred into a solution of cyclopropyl magnesium bromide in THF at 0-5° C., followed by immediate reduction with lithium aluminum hydride. Hydroiodic acid is added, the mixture is heated to give 4a after purification.

Step 4B:

Compound 4b (synthesized according to various procedures in U.S. Pat. No. 6,531,475) is alkylated with compound 4a using standard conditions such as sodium hydride, sodium methoxide or cesium carbonate in a solvent such as DMF followed by purification by HPLC to give 4-1.

### Example 5

### CRF Receptor Binding Activity

The compounds of this invention may be evaluated for binding activity to the CRF receptor by a standard radioligand binding assay as generally described by Grigoriadis et al. (Mol. Pharmacol vol 50, pp 679-686, 1996) and Hoare et al. (Mol. Pharmacol vol 63 pp 751-765, 2003.) By utilizing radiolabeled CRF ligands, the assay may be used to evaluate the binding activity of the compounds of the present invention with any CRF receptor subtype.

Briefly, the binding assay involves the displacement of a radiolabeled CRF ligand from the CRF receptor. More specifically, the binding assay is performed in 96-well assay plates using 1- 10 μg cell membranes from cells stably transfected with human CRF receptors. Each well receives about 0.05 ml assay buffer (e.g., Dulbecco's phosphate buffered saline, 10 mM magnesium chloride, 2 mM EGTA) containing compound of interest or a reference ligand (for example, sauvagine, urocortin I or CRF), 0.05 ml of [¹²⁵I] tyrosine—sauvagine (final concentration ˜150 pM or approximately the K_{D} as determined by Scatchard analysis) and 0.1 ml of a cell membrane suspension containing the CRF receptor. The mixture is incubated for 2 hours at 22° C. followed by separation of the bound and free radioligand by rapid filtration over glass fiber filters. Following three washes, the filters are dried and radioactivity (Auger electrons from ¹²⁵I) is counted using a scintillation counter. All radioligand binding data may be analyzed using the non-linear least-squares curve-fitting programs Prism (GraphPad Software Inc) or XLfit (ID Business Solutions Ltd).

It will be appreciated that, although specific embodiments of the invention have been described herein for purposes of illustration, various modifications may be made without departing from the spirit and scope of the invention. Accordingly, the invention is not limited except as by the appended claims.

1. A compound taken from 2,5-dimethyl-3-(2,4-dimethoxyphenyl)-7-(diethylamino)pyrazolo[2,3-a]pyrimidine, 2,5-dimethyl-3-(2,4-dimethoxyphenyl)-7-(N-ethyl-N-methoxyethylamino)pyrazolo[2,3-a]pyrimidine, 2,5-dimethyl-3-(2,4-dimethoxyphenyl)-7-{2-(S)-methoxymethylpyrrolidinyl}-pyrazolo[2,3-a]pyrimidine, and 2,5-dimethyl-3-(2,4-dimethoxyphenyl)-7-(N-ethyl-N-{2-fluoroethyl}amino)pyrazolo[2,3-a]pyrimidine.

2. A compound taken from 2,5-dimethyl-3-(2,4-dimethoxyphenyl)-7-(diethylamino)pyrazolo[2,3-a]pyrimidine, 2,5-dimethyl-3-(2,4-dimethoxyphenyl)-7-(N-ethyl-N-methoxyethylamino)pyrazolo[2,3-a]pyrimidine, 2,5-dimethyl-3-(2,4-dimethoxyphenyl)-7-{2-(S)-methoxymethylpyrrolidinyl}-pyrazolo[2,3-a]pyrimidine, 2,5-dimethyl-3-(2,4-dimethoxyphenyl)-7-(N-ethyl-N-{2-fluoroethyl}amino)pyrazolo[2,3-a]pyrimidine and 6-(cyclopropylmethyl)-2-(2,4-dichlorophenyl)-7-(S)-ethyl-7,8-dihydro-4-methyl-6H-1,3,6,8a-tetraazaacenaphthylene for use as PET ligands.

3. A composition comprising a compound of claim 1 in combination with a pharmaceutically acceptable carrier or diluent.

4. A method for treating a disorder manifesting hypersecretion of CRF in a warm-blooded animal comprising administering to the animal an effective amount of the pharmaceutical composition of claim 3.

5. The method of claim 4 wherein the disorder is depression.

6. The method of claim 4 wherein the disorder is an anxiety-related disorder.

7. The method of claim 4 wherein the disorder is irritable bowel syndrome.

8. A composition comprising a compound of claim 2 in combination with a pharmaceutically acceptable carrier or diluent.
